(19) United States
(12) Patent Application Publication
(10) Pub. No.: US 2003/0125515 A1
(43) Pub. Date: Jul. 3, 2003

(54) End-locked five-helix protein

(76) Inventor: Genfa Zhou, Chestnut Hill, MA (US)

Correspondence Address: Genfa Zhou, 21 Sherman Rd., Chestnut Hill, MA, 02467, US

(21) Appl. No.: 10/193,412

(22) Filed: Jul. 11, 2002

Related U.S. Application Data
(60) Provisional application No. 60/304,152, filed on Jul. 11, 2001.

(51) Int. Cl. A61K 39/21; C07K 14/16; C07H 21/04; A61K 39/12; C07K 2/00; C07K 4/00; C07K 5/00; C07K 7/00; C07K 14/00; C07K 16/00; C07K 17/00; A61K 38/00; C07K 1/00

(52) U.S. Cl. 530/300; 530/350; 424/188.1; 536/23.72; 424/204.1

(57) ABSTRACT

End-locked five-helix protein, which is made up of three N-helices and two C-helices of HIV gp41, four inside linkers, and at least one terminal linker; the helices are connected by the inside linkers, and the terminal linker is connected to an helix and is capable of cross-linking with one of the inside linkers, is disclosed.

### RELATED APPLICATION

This application claims the benefit of U.S. Provisional Application No. 60/304,152, entitled “Inhibitors of Viral Membrane Fusion” by Genfa Zhou (filed Jul. 11, 2001). The entire teachings of the referenced provisional applications are incorporated herein by reference.

### SEQUENCE LISTING

SEQ ID NO.: 1, SEQ ID NO.: 2, SEQ ID NO.: 3, SEQ ID NO.: 4, SEQ ID NO.: 5, SEQ ID NO.: 6, SEQ ID NO.: 7, SEQ ID NO.: 8, SEQ ID NO.: 9, SEQ ID NO.: 10, SEQ ID NO.: 11, SEQ ID NO.: 12, SEQ ID NO.: 13, SEQ ID NO.: 14, SEQ ID NO.: 15, SEQ ID NO.: 16, SEQ ID NO.: 17.

### BACKGROUND OF THE INVENTION

The human immunodeficiency virus (HIV) destroys the immune system and leaves victims vulnerable to an array of opportunistic infections and aggressive cancers. HIV infection results in AIDS, the leading cause of death in Africa and the fourth leading cause of death globally.

The initial steps of infection by enveloped viruses, such as HIV viruses and Ebola viruses include the attachment of viruses to host cells and fusion of viral and cellular membranes, which is mediated by envelope glycoproteins on the surfaces of the viruses. These envelope glycoproteins, also known as fusion proteins, including gp41 for HIV virus and GP2 for Ebola virus, are critical for membrane fusion. As seen in gp41 of HIV (FIG. 1), the ectodomains of the fusion proteins are usually rod-shaped α-helical trimers composed of three identical subunits. While each subunit of the trimer contributes to one inner peptide and one outer peptide, the three subunits form a bundle of six helices. The core of the bundle is a parallel, triple-stranded, α-helical coiled-coil, made up of the N-terminal α-helices (N-helices). Whereas wrapped antiparallelly on the outside of this core is an outer layer of three C-terminal α-helices (C-helices). This structural feature is shared by many fusion-mediating glycoproteins of enveloped viruses, including the gp41 subunit of HIV virus, the GP2 subunit of Ebola virus, the HA2 subunit of influenza virus, the F1 subunit of the paramyxovirus SV5, and the TM subunits of the retroviruses MuMoLV, HTLV, and SIV. In all these cases, the fusion glycoproteins form a rod-shaped α-helical bundle (reviewed by Skehel and Wiley, Cell 95, 871 (1998)).

When HIV virus infects cells, the conformation of HIV gp41 changes: the N-helix region of gp41 inserts into the cellular membrane, while the C-helix region of gp41 remains in the viral membrane. Through the rearrangement, gp41 brings together the cellular membrane and viral membrane, and makes the membrane fusion, the first step of HIV infection possible. It is therefore believed that disruption of gp41 function in membrane fusion would in turn inhibit HIV infection of host cells.

Various strategies have been developed to disrupt gp41 function in membrane fusion. One approach is focused on the development of C-peptides, corresponding to C-helix region of gp41, as potential inhibitors of membrane fusion (Jiang, S., et al., Nature 365, 113 (1993), and Wild, C. T., et al., Proc. Natl. Acad. Sci. U.S.A. 91, 9770 (1994)). It is suggested that C-peptides inhibit membrane fusion through binding to the N-peptide region of gp41 in a dominant-negative fashion. One of the C-peptides, T-20, is currently tested in clinical trials and has been shown antiviral activity in humans (Kilby, J. M., et al., Nature Med. 4, 1302 (1998)). However, C-peptides are too short to be produced using genetic engineering techniques, so that production of C-peptides poses significant manufacturing challenges. In addition, C-peptides are vulnerable to proteolytic digestion in human bodies. Therefore, if used as fusion inhibitors, C-peptides face stability and manufacturing challenges. On the other hand, N-peptides, corresponding to N-helix region of gp41, not only have the same stability and manufacturing problems, but also have the obstacle of insolubility. If used as drug targets for small molecule inhibitors, neither C-peptides nor N-peptides are useful, because neither has defined structure by itself.

Hence, there is a need to develop stable and soluble proteins to be used as inhibitors capable of blocking the membrane fusion, as drug targets for screening small molecule inhibitors, or as immunogens with well-defined and intact epitopes for vaccine development.

### SUMMARY OF THE INVENTION

The present invention provides an end-locked five-helix protein, which is made up of three N-helices and two C-helices of HIV gp41, four inside linkers, and at least one terminal linker; the helices are connected by the inside linkers, and the terminal linker is connected to an helix and is capable of cross-linking with one of the inside linkers.

In a preferred embodiment of the present invention, the protein is made up of, from N-terminus to C-terminus, an N-terminal linker, a first N-helix, a first inside linker, a first C-helix, a second inside linker, a second N-helix, a third inside linker, a second C-helix, a fourth inside linker, a third N-helix, and a C-terminal linker; wherein the N-terminal linker cross-links with the second inside linker or the fourth inside linker, and the C-terminal linker cross-links with the first inside linker or the third inside linker.

According to an embodiment of the present invention, both the inside linkers and terminal linkers are made up of amino acid residues.

According to an embodiment of the present invention, the terminal linkers of the five-helix protein cross-link with the inside linkers through the interaction of side chains of amino acid residues.

According to another embodiment of the present invention, a terminal linker of the end-locked five-helix protein cross-links with an inside linker through a covalent bond.

According to a further embodiment of the present invention, the covalent bond that cross-links a terminal linker of the five-helix protein with an inside linkers is a disulfide bond.

The present invention further provides an isolated protein having a sequence selected from the group consisting of SEQ ID NO.: 1, SEQ ID NO.: 2, SEQ ID NO.: 3, and SEQ ID NO.: 4.

The present invention also provides an isolated polynucleotide encoding the protein having a sequence selected from the group consisting of SEQ ID NO.: 1, SEQ ID NO.: 2, SEQ ID NO.: 3, and SEQ ID NO.: 4.

An embodiment of the present invention is an end-locked five-helix protein, which is made up of, from N-terminus to C-terminus, a first N-helix, a first inside linker, a first C-helix, a second inside linker, a second N-helix, a third inside linker, a second C-helix, a fourth inside linker, a third N-helix, and an C-terminal linker; the C-terminal linker cross-links with the first inside linker or the third inside linker.

An embodiment of the present invention is an end-locked five-helix protein, which is made up of, from N-terminus to C-terminus, an N-terminal linker, a first N-helix a first inside linker, a first C-helix, a second inside linker, a second N-helix, a third inside linker, a second C-helix, and a fourth inside linker, a third N-helix; the N-terminal linker cross-links with the second inside linker or the fourth inside linker.

The present invention also provides a method of inhibiting the entry of HIV into a cell, the method comprising contacting HIV with the end-locked five-helix protein.

An embodiment of the present invention is a method of inhibiting the entry of HIV into a human cell.

The present invention further provides a method of inhibiting HIV infection in a host, the method comprising administering to the host a composition comprising the end-locked five-helix protein.

An embodiment of the present invention is a method of inhibiting HIV infection in human, the method comprising administering to human a composition comprising the end-locked five-helix protein.

The present invention also provides a method of eliciting an immune response to HIV in a host, the method comprising introducing into the host a composition comprising the end-locked five-helix protein.

The present invention further provides a method of identifying a compound that inhibits HIV infection, the method comprising contacting the end-locked five-helix protein with the compound.

An embodiment of the present invention is a method of identifying a compound that inhibits HIV infection, the method comprising determining whether the compound interferes the association of labeled-C-peptide region of HIV gp41 and end-locked five-helix protein and determining whether the compound inhibits HIV infection of mammalian cells.

According to a further embodiment of the present invention, the labeled-C-peptide region of HIV gp41 is fluorescence-labeled.

### BRIEF DESCRIPTION OF THE DRAWINGS

FIG. 1 illustrates the structure of the ectodomain of HIV gp41. Three inner peptides (also known as N-peptides, or N-helices) in the shape of parallel α-helical coiled-coil form the inner core. The three α-helical outer peptides (also known as C-peptides, or C-helices) wrap around the inner core in an antiparallel manner.

FIG. 2a and 2b depict the design and diagram of a five-helix protein. FIG. 2a is the schematic design for a five-helix protein. The core structure has three N-helices helices (inner) and two C-helices (outer) joined by four inside linkers. FIG. 2b is the side and top view of a diagram of a five-helix protein. The dotted line indicates the missing third C-helix comparing to the wild-type gp41. The missing C-helix opens up the binding side on the inner core of the α-helical bundle. It makes possible for the five-helix protein to function as a mimic of HIV gp41 inner core.

FIGS. 3a-3d demonstrate the end lock of the five-helix proteins through disulfide bounds (S-S). FIG. 3a shows the introduction of two cysteins on each end for five-helix proteins with one pair from N-terminus and inside linker 2, and the other pair from C-terminus and inside linker 1. FIG. 3b shows the introduction of two cysteins on each end for five-helix proteins with one pair from N-terminus and inside linker 2, and the other pair from C-terminus and inside linker 3. FIG. 3c shows the introduction of two cysteins on each end for five-helix proteins with one pair from N-terminus and inside linker 4, and the other pair from C-terminus and inside linker 1. FIG. 3d shows the introduction of two cysteins on each end for five-helix proteins with one pair from N-terminus and inside linker 4, and the other pair from C-terminus and inside linker 3.

FIG. 4 is the schematic primer design for the construction of an end-locked five-helix protein, which corresponds to FIG. 3b (SEQ ID NO.: 2).

FIG. 5 shows the size exclusion profile of the end-locked five-helix protein with molecular weight of 23.4 kD eluted out from a Superdex-75. It shows the end-locked locked five-helix protein is soluble and is in right refolding based on the consistence between its molecular weight and the elution volume (13.5 ml).

FIG. 6 shows the circular dichroism (CD) spectrum of the end-locked five-helix protein. It shows the typical CD spectrum of alpha-helical structure, and demonstrates the end-locked five-helix protein is refolded correctly.

FIG. 7 shows the stability comparison between the end-locked five-helix protein and the wild-typed Gp41 (N36C34). The melting point Tm of the end-locked five-helix in PBS with 3 M GuHCl is 69° C., which is still much higher than the melting point 53° C. of gp41 (N36C34) in PBS only.

FIG. 8 shows the HIV virus inhibitory activity of the end-locked five-helix protein. According to the virus-cell fusion assay, the end-locked five-helix, derived from HXB2 (CXCR4), has greater inhibitory activity against HIV JRFL (CCR5) viruses with an EC₅₀ of 0.75 nM, while the EC₅₀ for inhibiting HIV HXB2 viruses is 15.03 nM. It is shown that the end-locked five-helix protein has potential inhibitory activity against very broad HIV strains.

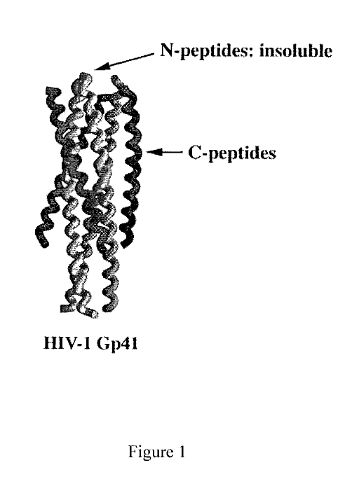

### DETAILED DESCRIPTION OF THE INVENTION

In addition to C-peptides, the inner cores of fusion proteins are also very attractive candidates for inhibitors and drug targets (Wild, C. T., et al., Proc. Natl. Acad. Sci. U.S.A. 89, 10537 (1992)). When binding to the C-peptide region of gp41, N-peptide should also be capable of inhibiting membrane fusion. However, the inner core itself formed by three inner peptides (N-peptide region) in the ectodomain without outer peptides (C-peptide region) is usually unstable and insoluble, which stymied the development of N-peptides as fusion inhibitors or as drug targets.

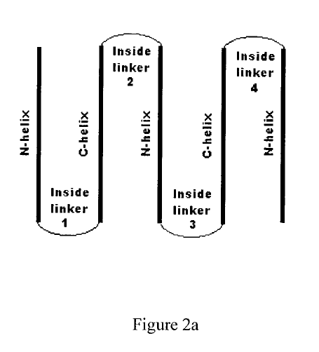

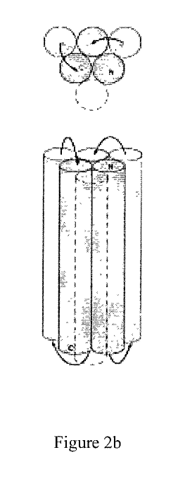

In order to make stable and soluble inner cores of fusion proteins, a five-helical bundle with three inner peptides (N-helices) and two outer peptides (C-helices) connected by linkers has been designed (Zhou, G., et al., unpublished data, and Root, M. J., et al, Science 291, 884 (2001), see FIG. 2a). The protein is referred as five-helix protein. The five-helix protein has the same inner core (made up of three N-helices) as wild type gp41, but has only two C-helices, and thereby creates a vacancy at one of C-helix position of gp41 (see FIG. 2b). The vacancy on the five-helix protein is a high-affinity binding site for the C-helix region of gp41. It is proposed that the five-helix protein can be used in different ways, including as inhibitors of membrane fusion, as HIV vaccine, as a tool for screening other inhibitors. (see, e.g., WO 01/44286 A2 by Root, M., et al.)

The data of preliminary analysis of the five-helix protein suggest that although it is more stable than N-helix by itself, the five-helix protein is still not stable enough for the desired utilities. For instance, antibodies elicited from five-helix protein have not shown any neutralization activity (Zhou, G., et al., unpublished data). Because it has its N-terminus and C-terminus open, the five-helix protein might be easily unfolded, and lose its conformation necessary for binding C-helix region of gp41, for binding of small molecule inhibitors, and for eliciting broad neutralization antibody against HIV infection. Hence, Applicant reasons that if its termini were locked, the stability and solubility of the five-helix protein would be improved significantly, and it would be more feasible for the proposed utilities.

The present invention provides an end-locked five-helix protein, which is made up of three N-helices and two C-helices of HIV gp41, four inside linkers, and at least one terminal linker; the helices are connected by the inside linkers, and the terminal linker is connected to an helix and is capable of cross-linking with one of the inside linkers.

As used herein, “N-helix”, and “C-helix” refer to the peptide sequences corresponding to the N-peptide portion and C-peptide portion of HIV gp41 subunit, respectively.

As used herein, “five-helix protein” refers to a genetically engineered protein including three N-helices and two C-helices that are linked together through inside linkers.

As used herein, “end-locked five-helix protein” refers to a five-helix protein that is connected to at least one terminal linker; the terminal linker cross-links with one of the inside linkers. The five-helix protein can be end-locked at either end, or at both ends.

In proteins, amino acids are joined together via peptide bond, which is formed by the reaction of α-carboxyl group of one amino acid with the α-amino group of another amino acid. As used herein, “cross-link” refers to a chemical bond that is not peptide bond. The chemical bond can be covalent bond, or ionic bond, or other bonds with similar strength.

As used herein, “inside linker” refers to the linker that connects two helices, preferably N-helix and C-helix.

As used herein, “terminal linker” refers to the linker that is connected to only one helix, preferably N-helix, and is capable of cross-linking to one of the inside linkers.

Both the inside linker and terminal linker can be of any length or composition, as long as the conformation of the end-locked five-helix protein is retained and the cross-link can be formed between the inside linker and terminal linker. The linkers are preferably composed of amino acid residues.

The amino acid sequence of the end-locked five-helix protein can be changed, so long as preserving a surface that is structurally complementary to the C-peptide region of HIV gp41 protein, and preferably binds to the C-peptide region. Such sequence changes may be needed to further increase the stability and solubility of the protein.

In a preferred embodiment of the present invention, the five-helix protein is made up of, from N-terminus to C-terminus, an N-terminal linker, a first N-helix, a first inside linker, a first C-helix, a second inside linker, a second N-helix, a third inside linker, a second C-helix, a fourth linker, a third N-helix, and a C-terminal linker; the N-terminal linker cross-links with the second inside linker or the fourth inside linker, and the C-terminal linker cross-links with the first inside linker or the third inside linker.

In a preferred embodiment of the present application, both the terminal linkers and the inside linkers are made up of amino acid residues. The length of the linkers can be from one amino acid residue to several amino acid residues, preferably more than one amino acid residue. In other words, the linkers are preferably peptides. The length of the terminal linker should be changed according to that of the inside linker that the terminal linker cross-links with. The lengths of the inside linker and the terminal linker should be long enough for a chemical bond to be formed between the linkers, while the conformation of the five-helix protein is retained. On the other hand, the lengths of the linkers should be short enough to “lock” the N-helix and C-helix in the antiparallel manner. If the lengths of the linkers are too long, the cross-link will not prevent the N-helix and C-helix from falling apart.

According to an embodiment of the present invention, the terminal linkers of the five-helix protein cross-link with the inside linkers through the interaction of side chains of amino acid residues. The cross-linked amino acid residues can be an amino acid residue in any position of either the terminal linker or the inside linker. Preferably, the cross-linked amino acid residue of the inside linker is located in the middle of the inside linker, while the cross-linked amino acid residue of the terminal linker is preferably the terminal amino acid residue. The terminal linker, as well as the corresponding inside linker, should have at least one amino acid residue with side chain capable of cross-linking.

The cross-link can also be formed between the side chain of an amino acid residue in the inside linker and the α-carboxyl group or α-amino group of the terminal amino acid residue in the terminal linker.

According to further embodiment of the present invention, a terminal linker of the end-locked five-helix protein cross-links with an inside linker through a covalent bond.

According to a preferred embodiment of the present invention, a terminal linker of the five-helix protein cross-links with an inside linker through a disulfide bond, a strong covalent bond. Formed between the side chains of two cystein residues, disulfide bond (S-S) is the most common type of cross-link of two different segments of a polypeptide, or two peptides. The thiol groups (SH) of the two cystein residues are oxidized to form a disulfide bond (S-S) so that two cystein residues are linked together as Cys-S-S-Cys, which is called cystine.

Disulfide bonds can be formed intramolecularly (i.e. within a single polypeptide chain) and intermolecularly (i.e. between two polypeptide chains). Intramolecular disulfide bonds stabilize the tertiary structures of proteins while intermolecular disulfide bonds are involved in stabilizing quaternary structures. The greater the number of disulfide bonds the less susceptible the protein to denaturation by other forces, such as detergents, heat, etc. Thus, it is expected that the introduction of disulfide bonds into the five-helix protein would increase both its in vivo stability and in vitro stability.

The formation of disulfide bonds occurs during the folding of proteins in the endoplasmic reticulum (ER) of eukaryotes and the periplasmic space of prokaryotes. Thiol disulfide exchange and disulfide bond formation are catalyzed by thiol-disulfide oxidoreductases. In eukaryotes, this process is catalyzed by protein disulfide isomerase (PDI), and in prokaryotes, by dsbA. The active sites of both PDI and dsbA share homology with the active site of thioredoxin.

For a disulfide bond to form, the redox environment must be oxidizing. Eukaryotic ER is more oxidizing than the surrounding cytosol. This is maintained by the high ratio of oxidized glutathione to reduced glutathione. In eukaryotes, therefore, most secreted proteins are disulfide bonded whereas most cytosolic proteins are not. Cystein residues in extracellular proteins are often cross-linked with disulfides as there is no reducing potential to keep the sulfur atoms reduced. For its utilities, the end-locked five-helix protein will be either used in extracellular conditions, or in vitro. Hence, the disulfide bond will be maintained to keep the five-helix protein end-locked and stable under the conditions of the utilities.

Various oxidative reagents, such as oxygen (air), dimethyl sulphoxide, oxidized glutathione, potassium ferricyanide, thallium(III) trifluro aectate, have been used to accomplish such a thiol-disulfide conversion. The oxidation of cysteine residues and the formation of disulfide bond can therefore be catalyzed in vitro by atmospheric oxygen. On the other hand, although it is a strong covalent bond, disulfide bond can be broken by appropriate reducing reagents, such as β-mercaptoethanol (HSCH2CH2OH). To inhibit the formation of disulfide bond in purified proteins, reducing reagents, such as ascorbic acid (vitamin C) or dithioerthritol (DTT), can be added to keep cysteins from being oxidized by atmospheric oxygen. The formation of the disulfide bonds in the five-helix protein can therefore be controlled with appropriate oxidative reagents and reducing reagents.

As described herein, based on the structural information of the rod-shaped fusion proteins, two cysteins on each end are introduced to form disulfide bonds to make both N-terminus and C-terminus locked in various ways. The introduction of cysteins can be achieved in, but not limited to, the following preferred designs.

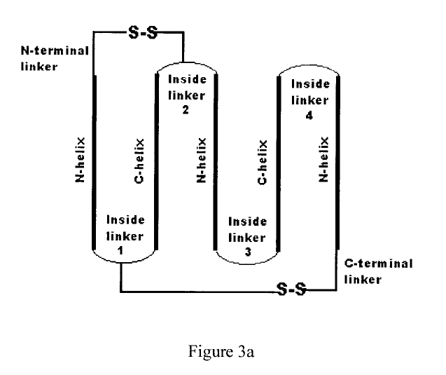

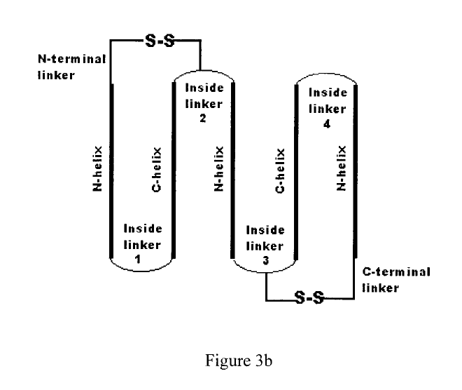

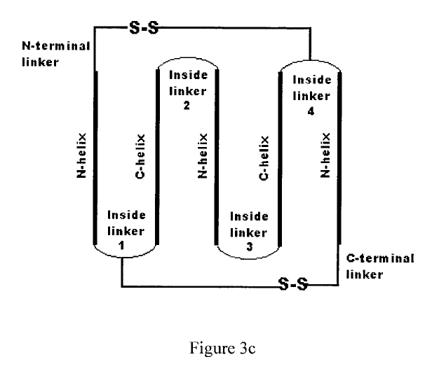

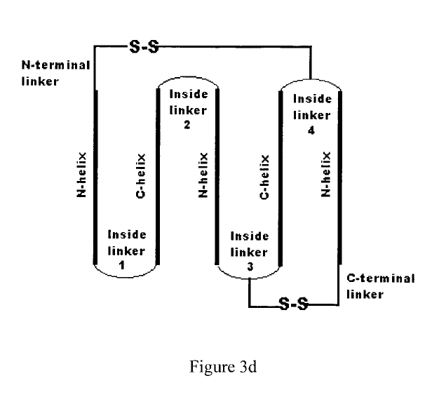

As shown in FIG. 3a, on one end (top) of an end-locked five-helix protein, the cystein on N-terminal linker is paired with the cystein on inside linker 2. On the other end (bottom), the cystein on C-terminal linker is paired with the cystein on inside linker 1.

According to one embodiment of the present application, the end-locked five-helix protein has a sequence as set forth in SEQ ID NO.: 1, and is as follows:

(SEQ ID NO.:1)

MCGGGSQLLS GIVQQQNNLL RAIEAQQHLL QLTVWGIKQL
QASGGCGGSW MEWDREINNY TSLIHSLIEE SQNQQEKNEQ
ELLSGGCGGS QLLSGIVQQQ NNLLRAIEAQ QHLLQLTVWG
IKQLQASGGS GGSWMEWDRE INNYTSLIHS LIEESQNQQE
KNEQELLSGG SGGSQLLSGI VQQQNNLLRA IEAQQHLLQL
TVWGIKQLQA SGGGC

As shown in single letter code, the underlined portion represents the terminal linkers and the inside linkers.

As shown in FIG. 3b, on one end (top) of an end-locked five-helix protein, the cystein on N-terminal linker is paired with the cystein on inside linker 2. On the other end (bottom), the cystein on C-terminal linker is paired with the cystein on inside linker 3.

According to one embodiment of the present application, the end-locked five-helix protein has a sequence as set forth in SEQ ID NO.: 2, and is as follows:

(SEQ ID NO.:2)

MCGGGSQLLS GIVQQQNNLL RAIEAQQHLL QLTVWGIKQL
QASGGSGGSW MEWDREINNY TSLIHSLIEE SQNQQEKNEQ
ELLSGGCGGS QLLSGIVQQQ NNLLRAIEAQ QHLLQLTVWG
IKQLQASGGC GGSWMEWDRE INNYTSLIHS LIEESQNQQE
KNEQELLSGG SGGSQLLSGI VQQQNNLLRA IEAQQHLLQL
TVWGIKQLQA SGGGC

As shown in single letter code, the underlined portion represents the terminal linkers and the inside linkers.

As shown in FIG. 3c, on one end (top) of an end-locked five-helix protein, one cystein on N-terminal linker is paired with one cystein on inside linker 4. On the other end (bottom), one cystein on C-terminal linker is paired with one cystein on inside linker 1.

According to one embodiment of the present application, the end-locked five-helix protein has a sequence as set forth in SEQ ID NO.: 3, and is as follows:

(SEQ ID NO.:3)

MCGGGSQLLS GIVQQQNNLL RAIEAQQHLL QLTVWGIKQL
QASGGCGGSW MEWDREINNY TSLIHSLIEE SQNQQEKNEQ
ELLSGGSGGS QLLSGIVQQQ NNLLRAIEAQ QHLLQLTVWG
IKQLQASGGS GGSWMEWDRE INNYTSLIHS LIEESQNQQE
KNEQELLSGG CGGSQLLSGI VQQQNNLLRA IEAQQHLLQL
TVWGIKQLQA SGGGC

As shown in single letter code, the underlined portion represents the terminal linkers and the inside linkers.

As shown in FIG. 3d, on one end (top) of an end-locked five-helix protein, one cystein on N-terminal linker is paired with one cystein on inside linker 4. On the other end (bottom), one cystein on C-terminal linker is paired with one cystein on inside linker 3.

According to one embodiment of the present application, the end-locked five-helix protein has a sequence as set forth in SEQ ID NO.: 4, and is as follows:

(SEQ ID NO.:4)

MCGGGSQLLS GIVQQQNNLL RAIEAQQHLL QLTVWGIKQL
QASGGSGGSW MEWDREINNY TSLIHSLIEE SQNQQEKNEQ
ELLSGGSGGS QLLSGIVQQQ NNLLRAIEAQ QHLLQLTVWG
IKQLQASGGC GGSWMEWDRE INNYTSLIHS LIEESQNQQE
KNEQELLSGG CGGSQLLSGI VQQQNNLLRA IEAQQHLLQL
TVWGIKQLQA SGGGC

As shown in single letter code, the underlined portion represents the terminal linkers and the inside linkers.

The two cysteins on each end with flexible inside linker or terminus are close enough to form covalently linked disulfide bond to make the ends locked. Because of the flexibility and the lengths of the inside linker and the terminal linker, the formation of the disulfide bond should not change the conformation of the protein. In addition, the lengths of the linkers are not too long to “lock” the N-helix and C-helix together. Therefore, the end-locked five-helical bundle will be able to stay folded to significantly increase its stability and solubility.

A protein containing disulfide bonds can be first synthesized with cysteins in its sequence via genetic engineering techniques, then, followed by the oxidation of the thiol groups to form disulfide bonds. If there is only one pair of cysteins, the formation of disulfide bond is pretty straightforward. If there is more than one pair of cysteins, the formation of disulfide bonds becomes complicated. If both ends of the five-helix protein are to be locked, two pairs of cysteins are needed. The mismatch of the cysteins can be avoided as follows. The protein can be synthesized, and unfolded with urea and a reducing reagent such as DTT, which keeps cysteins from being oxidized by atmospheric oxygen. After removing the urea by dialysis, direct dilution, or gel filtration, the protein is refolded, and then the disulfide bonds are formed, since each pair of two cysteins are close enough on each end of the rod-shaped protein. With the correct folding of the five-helix protein and the linkers in proper lengths, only the desired disulfide bonds will be formed, as illustrated in FIG. 3a-3d, and the protein will be correctly end-locked.

The oxidation of cysteins and the formation of the disulfide bond can be monitored by Ellman Test for the amount of thiol group left and by RP-HPLC (Reversed Phase Chromatography) for the progress of oxidation.

The genes encoding the above designed proteins will be cloned by using conventional technique and expressed in bacteria, insect cells, yeast, or mammalian cells, either as soluble proteins or as inclusion bodies. If they are expressed as inclusion bodies, the inclusion bodies will be solubilized in 8M urea or 6M Guanidine Hydrochloride and 5 mM DTT, and are refolded by direct dilution, gradually dialysis, or gel filtration. The disulfide bond can be formed only after correct and stable protein is refolded. Iron salts may be added to facilitate the formation of the disulfide bonds within the protein.

The cross-link in the end-locked five-helix protein can also be covalent bond other than disulfide bond. For instance, cross-link occurs intramolecularly within tropocollagen molecule, where two Lysine residues are oxidized to allysine, an aldehyde, and then condensed to form lysinonorleucine (forms in the non-helical N-terminal regions of tropocollagen). The oxidation of lysine is catalyzed by lysyl oxidase. With the introduction of lysines into terminal linker and corresponding inside linker, such a cross-link can be used to end-lock the five-helix protein in appropriate conditions and stabilize the protein.

The cross-link between amino acid residues other than cysteine might not be formed spontaneously. After the five-helix proteins are synthesized, further treatment with chemical reagents and enzymes might be needed to form the cross-link and end-lock the protein.

These genetically engineered proteins can be utilized in, but not limited to, the following ways.

The end-locked five-helix protein can be used as fusion inhibitors to block viral entry. It has demonstrated that end-locked five-helix protein can inhibit the membrane fusion of HIV virus and the host cells (see EXAMPLE 3). These genetically engineered proteins are stable and soluble so that the proteins themselves can be used as drug candidates to block viral membrane fusion. These proteins will be administered through injection.

The end-locked five-helix protein can be used as vaccine. These engineered proteins have the same highly conserved epitopes as the natural viral fusion proteins. Antibodies elicited by the administration of the engineered proteins will attack the natural viral surface proteins to defend host cells from the viral infection.

The end-locked five-helix protein can also be used as tools for screening drug candidates against HIV. These soluble engineered proteins can be used as drug targets for identifying and designing drugs or agents that inhibit entry of viruses into cells. For example, these soluble engineered proteins can be used as targets for small molecule inhibitors by high throughput library screening. A labeled C-peptide of HIV gp41 can be engineered, for instance, fluorescence is labeled on one end of the C-peptide. By monitoring the interference of the association of the labeled C-peptide and the end-lock five-helix protein, small molecule leads can be found by screening a large small molecule library.

Infections of humans with the filoviruses, Ebola virus, and Marburg virus are rare, but the resulting hemagorrhagic fevers are associated with high mortality. Four subtypes of Ebola viruses, Zaire, Sudan, Ivory Coast, and Reston, have been identified by genome sequencing. Their single envelope glycoproteins (GP) are synthesized as single-chain precursors and transferred cotranslationally into the lumen of the endoplasmic reticulum where they form trimers. Then they are post-translationally cleaved into two chains, GP1 and GP2, analogous to gp120 and gp41 of HIV. The fusion subunit ectodomain of GP2 is a trimer with highly α-helical, rod-shaped conformation. However, the inner core of GP2 itself is not stable. The same strategy used in the design of inhibitor for HIV gp41 can be applied to design stable and soluble inhibitors for GP2, which mimic the inner core of GP2. These engineered proteins can be useful as inhibitors to block Ebola viruses, as vaccine to induce immune response against Ebola viral infection, and as drug targets for identifying and designing drugs or agents which inhibit entry of Ebola viruses into cells.

The same strategy described above can also be applied to the drug design for the HA2 subunit of influenza virus, the F1 subunit of the paramyxovirus SV5, and the TM subunits of the retroviruses MuMoLV HTLV and SIV. In all these cases, the fusion glycoprotein forms a rod-shaped α-helical bundle. Both N-terminus and C-terminus of the five-helical bundle can be locked to make stable and soluble mimics of its corresponding inner core by the introduction of cysteins for the formation of disulfide bonds.

The following examples are provided by the way of illustration and are not intended to limit the scope of the present invention.

### EXAMPLE 1

### The Making of the End-locked Five-helix Protein

The end-locked five-helix protein having SEQ ID NO.: 2 (as shown in FIG. 3b) was produced in the procedures as follows.

1. Cloning of the Gene Encoding the Protein

There are three N-helices, and two C-helices, four inside linkers, and two terminal linkers in the end-locked five-helix protein. The gene encoding the end-locked five-helix protein was cloned with Polymerase Chain Reaction (RCR) using appropriate primers, and gp41 (HXB2) gene for original template as set forth in SEQ ID NO.: 5, and is as follows:

GCN4/gp41M (Weissenhorn, W., et al., Nature 387, 426 (1997), the loop sequence between N-peptide and C-peptide was mutated for other purposes):

(SEQ ID NO.:5)

CAT ATG AAA CAG ATC GAA GAC AAA ATC GAA
GAA ATC CTG TCC AAA ATC TAC CAC ATC GAA
AAC GAA ATC GCT CGT ATC AAA AAA CTG ATC
GGT GAA GCA CGC CAA TTA TTG TCT GGT ATA
GTG CAG CAG CAG AAC AAT TTG CTG AGG GCT
ATT GAG GCG CAA CAG CAT CTG TTG CAA CTC
ACA GTC TGG GGC ATC AAG CAG CTC CAG GCA
AGA ATC CTG GCT GTG GAA AGA TAC CTA AAG
GAT CAA CAG CTC CTG GGG ATT TGG GGT AGC
TCT GGT AAA CTG ATC AGC ACC ACT GCT CGC
CTG AGG AGG CTA GTC GCA GTG AGA AAT CTC
TGG AAC AGA TCT CTA GCT AGA CAC ACG ACC
TGG ATG GAG TGG GAC AGA GAA ATT AAC AAT
TAC ACA AGC TTA ATA CAC TCC TTA ATT GAA
GAA TCG CAA AAC CAG CAA GAA AAG AAT GAA
CAA GAA TTA TTG GAA TTA GAT AAA TGG GCA
AGT TTG TCG AAT TCG TTT AAC ATA ACA AAT
TAG CTG CAG CTC GTA CCA TGG AAT TCG AAG
CTT.

As shown above, the underlined portion represents the DNA sequences for N-helix and C-helix. Nde I (CATATG) and EcoR I (GAATTC) were used as restriction endonucleases.

The whole design and positions of the primers are shown in FIG. 4.

In first step, Sequences I-V were synthesized respectively by PCR using the template and primers as follows:

For Sequence I, the template was gp41 gene, and the primers were p1 and p2.

For Sequence II, the template was gp41 gene, and the primers were p3 and p4.

For Sequence III, the template was gp41 gene, and the primers were p5 and p6.

For Sequence IV, the template was gp41 gene, and the primers were p7 and p8.

For Sequence V, the template was gp41 gene, and the primers were p9 and p10.

In second step, Sequences VI and VII were synthesized respectively by PCR using the templates and primers as follows:

For Sequence VI, the template was Sequence I and Sequence II, and the primers were p1 and p4.

For Sequence VII, the template was Sequence III and Sequence IV, and the primers were p5 and p8.

In third step, Sequences VIII and IX were synthesized respectively by PCR using the templates and primers as follows:

For Sequence VIII, the template was Sequence VI and Sequence III, and the primers were p11 and p6.

For Sequence IV, the template was Sequence VII and Sequence V, and the primers were p5 and p12.

Finally, the gene encoding the end-locked five-helix protein was synthesized by PCR using the Sequence VIII and Sequence IV as templates, and p11 and p12 as primers.

The DNA sequences of the primers used for the construction of the gene encoding the end-locked five-helix protein, set forth as SEQ ID NO.: 6-17, were as follows:

| | |
|---|---|
| Primer P1: | |
| TGA CTG TGG GAA TTC CAT ATG TGT | (SEQ ID NO.:6) |
| GGA GGC GGA AGC CAA TTA TTG TCT | |
| CGT ATA | |
| Primer P2: | |
| ACT GCC TCC TGA GCC ACC AGA TGC | (SEQ ID NO.:7) |
| CTG GAG CTG CTT GAT | |
| Primer P3: | |
| TCT GGT GGC TCA GGA GGC AGT TGG | (SEQ ID NO.:8) |
| ATG GAG TGG GAC AGA | |
| Primer P4: | |
| GGA CCC TCC ACA GCC ACC ACT CAA | (SEQ ID NO.:9) |
| TAA TTC TTG TTC ATT | |
| Primer P5: | |
| AGT GGT GGC TGT GGA GGG TCC CAA | (SEQ ID NO.:10) |
| TTA TTG TCT GGT ATA | |
| Primer P6: | |
| TGA ACC GCC GCA TCC CCC GCT TGC | (SEQ ID NO.:11) |
| CTG GAG CTG CTT GAT | |
| Primer P7: | |
| AGC GGG GGA TGC GGC GGT TCA TGG | (SEQ ID NO.:12) |
| ATG GAG TGG GAC AGA | |
| Primer P8: | |
| ACT TCC GCC GCT TCC CCC CGA CAA | (SEQ ID NO.:13) |
| TAA TTC TTG TTC ATT | |
| Primer P9: | |
| TCG GGG GGA AGC GGC GGA AGT CAA | (SEQ ID NO.:14) |
| TTA TTG TCT GGT ATA | |
| Primer P10: | |
| GGA TCA AGC TTC GAA TTC TTA ACA | (SEQ ID NO.:15) |
| ACC GCC TCC ACT TGC CTG GAG CTG | |
| CTT GAT | |
| Primer P11: | |
| TGA CTG TGG GAA TTC CAT ATG TGT | (SEQ ID NO.:16) |
| GGA GGC GGA AGC | |
| Primer P12: | |
| GGA TCA AGC TTC GAA TTC TTA ACA | (SEQ ID NO.:17) |
| ACC GCC TCC ACT | |

2. Protein Expression

The DNA fragment encoding the end-locked five-helix was subcloned into the expression vector pRSET (Invitrogen) and transformed into Escherichia coli cells BL21 DE3/pUBS. After 0.1 mM IPTG induction at A₅₉₅=0.5 in media of LB w/Amp at 37° C., the culture was incubated at 37° C. for another 3 hr. The cells were pelleted down at 5000 rpm for 15 min; the supernatant was discarded, and the pellet was stored at −20° C.

3. Protein Unfolding

Bacteria were lysed in PBS by sonication, and insoluble material was pelleted down at 6000 rpm (T.45 rotor, Beckman) for 40 min. Inclusion bodies were purified by washing the pellet three times with PBS/0.5% Triton X-100 and once in PBS without Triton X-100, and then solubilized in 8 M urea with 5 mM DTT. The dissolved solution in 8 M urea and 5 mM DTT was diluted at the final concentration of 1 mg/ml and was totally unfolded by heating at 100° C. for 30 min, followed by room temperature for 30 min.

3. Purification/Refolding and End-Locking of the Protein

The protein was refolded and purified by gel filtration chromatography with Superdex-75 (Pharmacia) in PBS, pH7.0. Through the gel filtration chromatography, the protein buffer can be exchanged. With the removal of urea, the protein was refolded. The disulfide bonds were formed spontaneously between a terminal linker and an inside linker of each end of the protein only after the special rod-shaped protein was refolded correctly.

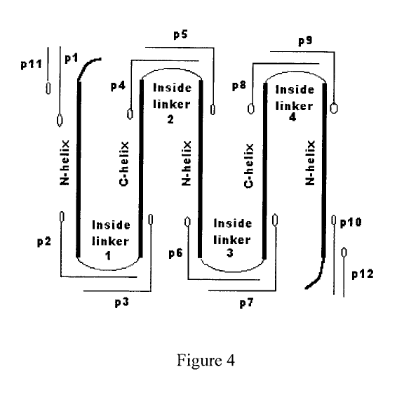

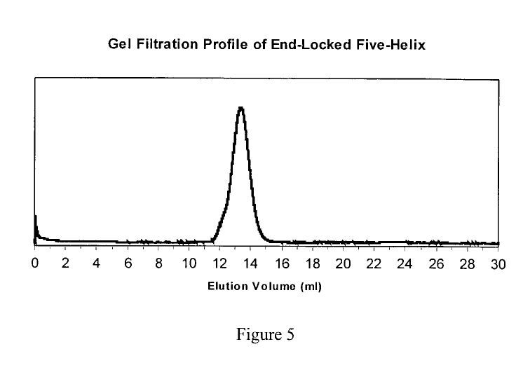

The profile is shown as FIG. 5. The peak came at 13.5 ml, which was consistent with the calculated elution volume based on the molecular weight of 23.4 kD for the end-locked five-helix protein. It was shown that the end-locked five-helix protein was soluble and was refolding right. The final product was therefore the five-helix protein having a sequence as shown in SEQ ID NO.: 2, with its ends locked by the disulfide bonds, as illustrated in FIG. 3b.

### EXAMPLE 2

### The Stability Comparison of End-locked Five-helix and GP41

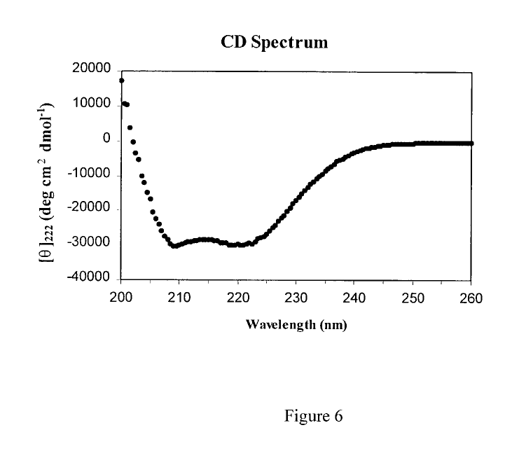

The CD spectrum (see FIG. 6) of end-locked five-helix protein (1 mg/ml) in PBS, pH 7.0, was recorded at 20° C. using a 1 mm cell on an AVIV 623DS spectropolarimeter with a thermoelectric controller. Ten independent measurements were averaged in the range 200-260 nm. It was shown with a typical CD spectrum for alpha-helical structure. That demonstrated the end-locked five-helix protein is refolded as alpha-helical bundle as expected.

Thermodynamic stability was measured at 222 nm by monitoring the CD signal in the range 25-95° C. at a concentration of 0.4 mg/ml (see FIG. 7). The wild type six alpha-helical bundle of Gp41 ectodomain (N36: residues 35-70; C34: residues 117-150) was measured with PBS, pH7.0, and end-locked five-helix was measured with PBS, pH7.0, containing 3 M GuHCl. The concentration was determined by measuring the OD280 with an extinction coefficient of 59 580/M/cm. The melting point Tm for gp41 in PBS is 53° C. However, The melting point of end-locked five-helix in PBS was only beyond the measuring range (higher than 95° C.). The Tm of end-locked five-helix in PBS and 3 M GuHCl is 69° C. that was still much higher than the Tm of gp41. Therefore, end-locked five-helix was much more stable than gp41. It is further demonstrated that the end-locked five-helix proteins were refolded correctly, and the disulfide bonds were formed.

### EXAMPLE 3

### Inhibition Activity of End-locked Five-helix Against HIV

Inhibitory activity of the end-locked five-helix protein was determined by using an HIV luciferase assay. Virus was made by cotransfecting 293T cells with an HIV-1 genome containing a frame-shift mutation in env and a luciferase gene replacing nef (NL43LucR-E-) along with pCMV-HXB2 or PEBB-JRFL, expression vectors with the HXB2 or the JRFL gp160 gene. Because its genome lacks env, the resultant virus is viable only for one round of infection. The cellular debris was removed by low-speed centrifugation. The remaining viral supernatant was used to infect HOS-CD4 (HXB2) cells or HOS-CD4-CCR5 (JRFL) cells (N. Landau, National Institutes of Health AIDS Reagent Program). Two days post-infection, the cells were lysed, and luciferase activity was monitored. IC₅₀ values (the concentration of the end-locked five-helix protein at which half of the viral infection is inhibited) were calculated by fitting the data to a Langmuir equation [y=k/(1+[end-locked five-helix]/IC₅₀)], where y−luciferase activity and k is a scaling constant.

We used the virus-cell fusion assay described above to measure the capacity of the end-locked five-helix protein to inhibit gp41-mediated cell membrane fusion (see FIG. 8). In the virus-cell fusion assay, the end-locked five-helix protein had an EC₅₀ of 15.03+/−1.41 nM and 0.75+/−0.11 nM for HIV HXB2 viruses (filled triangles) and HIV JRFL viruses (filled squares), respectively. The major obstacle for fighting HIV either through therapeutics or through vaccination is the development of resistance, which is due to the high mutation rates of most HIV proteins. The end-locked five-helix protein we used for the assay was derived from the sequence of HIV HXB2 (using CXCR4 as coreceptor). However, this end-locked five-helix protein has greater inhibitory activity against HIV JRFL viruses, which are using CCR5 as coreceptor. Therefore, the end-locked five-helix protein has the potential to fight against very broad HIV strains.

Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

I claim:

1. An end-locked five-helix protein comprising:
three N-helices and two C-helices of HIV gp41
four inside linkers, and
at least one terminal linker;
wherein the helices are connected by the inside linkers, and the terminal linker is connected to an helix and is capable of cross-linking with one of the inside linkers.

2. The protein according to claim 1 comprising from N-terminus to C-terminus:
an N-terminal linker, a first N-helix, a first inside linker, a first C-helix, a second inside linker, a second N-helix, a third inside linker, a second C-helix, a fourth inside linker, a third N-helix, and a C-terminal linker;
wherein the N-terminal linker cross-links with the second inside linker or the fourth inside linker, and the C-terminal linker cross-links with the first inside linker or the third inside linker.

3. The protein according to claim 2 wherein both the inside linkers and terminal linkers comprise amino acid residues.

4. The protein according to claim 3 wherein terminal linkers cross-link with the inside linkers through the interaction of side chains of amino acid residues.

5. The protein according to claim 4 wherein the terminal linkers cross-link with the inside linker through covalent bond.

6. The protein according to claim 5 wherein the covalent bond is a disulfide bond.

7. An isolated protein having a sequence selected from the group consisting of:
SEQ ID NO.: 1
SEQ ID NO.: 2
SEQ ID NO.: 3 and
SEQ ID NO.: 4.

8. An isolated polynucleotide encoding the protein according to claim 7.

9. The protein according to claim 1 comprising from N-terminus to C-terminus:
a first N-helix, a first inside linker, a first C-helix, a second inside linker, a second N-helix, a third inside linker, a second C-helix, a fourth inside linker, a third N-helix, and C-terminal linker;
wherein the C-terminal linker cross-links with the first inside linker or the third inside linker.

10. The protein according to claim 1 comprising from N-terminus to C-terminus:
an N-terminal linker, a first N-helix, a first inside linker, a first C-helix, a second inside linker, a second N-helix, a third inside linker, a second C-helix, and a fourth inside linker, a third N-helix;
wherein the N-terminal linker cross-links with the second inside linker or the fourth inside linker.

11. A method of inhibiting the entry of HIV into a cell, the method comprising contacting HIV with the protein according to claim 1.

12. The method according to claim 11, wherein the cell is a human cell.

13. A method of inhibiting HIV infection in a host, the method comprising administering to the host a composition comprising the protein according to claim 1.

14. The method according to claim 13, wherein the host is human.

15. A method of eliciting an immune response to HIV in a host, the method comprising introducing into the host a composition comprising the protein according to claim 1.

16. A method of identifying a compound that inhibits HIV infection, the method comprising contacting the protein according to claim 1 with the compound.

17. The method according to claim 16 further comprising determining whether the compound inhibits HIV infection of mammalian cells.


SEQUENCE LISTING (17 sequences)

SEQ ID NO: 1 - protein, 215 aa, Human immunodeficiency virus type 1
MCGGGSQLLSGIVQQQNNLLRAIEAQQHLLQLTVWGIKQLQASGGCGGSWMEWDREINNYTSLIHSLIEESQNQQEKNEQELLSGGCGGSQLLSGIVQQQNNLLRAIEAQQHLLQLTVWGIKQLQASGGSGGSWMEWDREINNYTSLIHSLIEESQNQQEKNEQELLSGGSGGSQLLSGIVQQQNNLLRAIEAQQHLLQLTVWGIKQLQASGGGC

SEQ ID NO: 2 - protein, 215 aa, Human immunodeficiency virus type 1
MCGGGSQLLSGIVQQQNNLLRAIEAQQHLLQLTVWGIKQLQASGGSGGSWMEWDREINNYTSLIHSLIEESQNQQEKNEQELLSGGCGGSQLLSGIVQQQNNLLRAIEAQQHLLQLTVWGIKQLQASGGCGGSWMEWDREINNYTSLIHSLIEESQNQQEKNEQELLSGGSGGSQLLSGIVQQQNNLLRAIEAQQHLLQLTVWGIKQLQASGGGC

SEQ ID NO: 3 - protein, 215 aa, Human immunodeficiency virus type 1
MCGGGSQLLSGIVQQQNNLLRAIEAQQHLLQLTVWGIKQLQASGGCGGSWMEWDREINNYTSLIHSLIEESQNQQEKNEQELLSGGSGGSQLLSGIVQQQNNLLRAIEAQQHLLQLTVWGIKQLQASGGSGGSWMEWDREINNYTSLIHSLIEESQNQQEKNEQELLSGGCGGSQLLSGIVQQQNNLLRAIEAQQHLLQLTVWGIKQLQASGGGC

SEQ ID NO: 4 - protein, 215 aa, Human immunodeficiency virus type 1
MCGGGSQLLSGIVQQQNNLLRAIEAQQHLLQLTVWGIKQLQASGGSGGSWMEWDREINNYTSLIHSLIEESQNQQEKNEQELLSGGSGGSQLLSGIVQQQNNLLRAIEAQQHLLQLTVWGIKQLQASGGCGGSWMEWDREINNYTSLIHSLIEESQNQQEKNEQELLSGGCGGSQLLSGIVQQQNNLLRAIEAQQHLLQLTVWGIKQLQASGGGC

SEQ ID NO: 5 - DNA, 543 bp, Human immunodeficiency virus type 1
catatgaaacagatcgaagacaaaatcgaagaaatcctgtccaaaatctaccacatcgaaaacgaaatcgctcgtatcaaaaaactgatcggtgaagcacgccaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggtagctctggtaaactgatcagcaccactgctcgcctgaggaggctagtcgcagtgagaaatctctggaacagatctctagctagacacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattagctgcagctggtaccatggaattcgaagctt

SEQ ID NO: 6 - DNA, 54 bp, Artificial
  Primer
tgactgtgggaattccatatgtgtggaggcggaagccaattattgtctggtata

SEQ ID NO: 7 - DNA, 39 bp, Artificial
  primer
actgcctcctgagccaccagatgcctggagctgcttgat

SEQ ID NO: 8 - DNA, 39 bp, Artificial
  primer
tctggtggctcaggaggcagttggatggagtgggacaga

SEQ ID NO: 9 - DNA, 39 bp, Artificial
  primer
ggaccctccacagccaccactcaataattcttgttcatt

SEQ ID NO: 10 - DNA, 39 bp, Artificial
  primer
agtggtggctgtggagggtcccaattattgtctggtata

SEQ ID NO: 11 - DNA, 39 bp, Artificial
  primer
tgaaccgccgcatcccccgcttgcctggagctgcttgat

SEQ ID NO: 12 - DNA, 39 bp, Artificial
  primer
agcgggggatgcggcggttcatggatggagtgggacaga

SEQ ID NO: 13 - DNA, 39 bp, Artificial
  primer
acttccgccgcttccccccgacaataattcttgttcatt

SEQ ID NO: 14 - DNA, 39 bp, Artificial
  primer
tcggggggaagcggcggaagtcaattattgtctggtata

SEQ ID NO: 15 - DNA, 54 bp, Artificial
  primer
ggatcaagcttcgaattcttaacaaccgcctccacttgcctggagctgcttgat

SEQ ID NO: 16 - DNA, 36 bp, Artificial
  primer
tgactgtgggaattccatatgtgtggaggcggaagc

SEQ ID NO: 17 - DNA, 36 bp, Artificial
  primer
ggatcaagcttcgaattcttaacaaccgcctccact